(19) United States
(12) Patent Application Publication
(10) Pub. No.: US 2010/0267126 A1
(43) Pub. Date: Oct. 21, 2010

(54) BIOREACTOR APPARATUS, BIOREACTOR SYSTEM, AND METHOD FOR GROWING LIGHT ENERGY DEPENDANT BIOLOGICAL SPECIES

(75) Inventors: Joseph Hendrik Anna Maria Jacobs, Eygelshoven (NL); Henricus Marie Peeters, Baarlo (NL); Denis Joseph Carel Van Oers, Bocholt (BE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/742,702

(22) PCT Filed: Nov. 17, 2008
(86) PCT No.: PCT/IB2008/054816

§ 371 Date: May 13, 2010

(30) Foreign Application Priority Data
Nov. 20, 2007 (EP) 07121089.2

(51) Int. Cl. C12M 1/00; A01G 9/18; A01K 61/00

(52) U.S. Cl. 435/292.1; 47/17; 119/200

(57) ABSTRACT

The invention relates to a bioreactor apparatus (1) for growing of biological species (2), comprising at least one basin device (3) defining a first habitat (4a) for a first species (2a), and a first lighting device (5a) having one light emitting solid-state lighting source (6), adapted for the first species (2a) by emitting light (L), wherein the solid-state lighting source (6) illuminates said habitat (4) using light energy emitted from the solid-state lighting source (6), wherein the bioreactor apparatus (1) comprises a second habitat (4b), adapted for a second species (2b). The invention further relates to a bioreactor system (20) comprising two bioreactor apparatus (1a,1b), one adopted as an aquatic ecoregion, one adopted as a terrestrial ecoregion, both combined to form a complex artificial ecoregion and a method for growing light energy dependant biological species (2) in one bioreactor apparatus (1), comprising: illuminating a first species (1) in a first habitat (4a) by a first lighting device (5a), transferring the grown first species (2a) to a successive habitat (4b, 4c, 4d, . . . ), separated from the previous habitat (4), via a connection system (7), illuminating a successive species (2b,2c, . . . ) in said successive habitat (4b,4c,4d, . . . ) by a successive lighting device (5b,5c, . . . ), and repeating the steps transferring and illuminating until the desired species (2) has grown to an optimum.

This invention relates to a bioreactor apparatus according to the preamble of claim 1, a bioreactor system comprising at least two a bioreactor apparatuses, and a method for growing light energy dependant biological species.

A bioreactor apparatus and a bioreactor system comprising a bioreactor apparatus are well known in the prior art.

For example, in U.S. Pat. No. 7,220,018 B2 a method and an apparatus of lighting a marine habitat for growth utilizing an LED light system are disclosed. The light system includes an LED light source, a power supply for such light source and a controller for controlling the activation status and the intensity of the LED light source. The apparatus comprises one single closed area, where the marine habitat is accommodated. The lighting source is adopted for one kind of species in one apparatus, only. So the apparatus according to U.S. Pat. No. 7,220,018 B2 is not suitable for more than one species, especially not for complete food chains.

Thus, the invention has been made in view of the above mentioned disadvantages. In particular, it is an object of the invention to provide an efficient apparatus, an efficient system, and an efficient method for an optimized growing of more than one light dependant species, which runs at a low cost.

The above and other objects can be achieved by an apparatus, a system and a method described in the independent claim. The dependent claims define further advantageous and exemplary combinations of the present invention.

The object of the invention is achieved by a bioreactor apparatus for growing of light energy dependant biological species, comprising at least one basin device defining a first habitat for accommodating a first species, and a first lighting device having at least one light emitting solid-state lighting source, adapted for the first species by emitting light, especially light having a spectrum set for the optimum growth of said first species, wherein the solid-state lighting source illuminates said habitat using light energy emitted from the solid-state lighting source to said first species, wherein the bioreactor apparatus comprises at least a second habitat, adapted for a second species, for growing at least two different kinds of species in one bioreactor system.

Light energy dependant biological species are all creatures needing light for growing. Examples of such light energy dependant biological species comprise: primary producers, autotrophs, consumers, heterotrophs, decomposers, detritivores, phytoplankton, zooplankton, algae, fish, nanoplankton, micorzooplankton, macrozooplankton, megazooplankton, zooplanktivorous fish, piscivorous fish, microphytoplankton, benthic herbivores, benthic carnivores, macrophytoplankton, planktivorous fish, megaplankton, planktivorous whales and the like.

A basin device in the sense of the invention comprises all housings, suitable for accommodating light energy dependant biological species. Preferably the basin device is a sealed basin device, especially an at least partly watertight basin device, an at least impermeable basin device, and/or an at least airtight basin device. The basin device may be designed as one single part or may be an arrangement of at least two parts secured together. In one preferred embodiment the basin device comprises a watertight basis and a top. The basis may be built watertight, having a bottom plate and a wall, defining an inside of the basin device. The top may be a lid, detachably arranged on the basis. In one preferred embodiment, the basin device is an aquarium. The basin device may comprise one single basin unit. Alternatively, the basin device may comprise at least two, preferable several basin units. The basin unit(s) may be arranged in the same way as the basin device.

The inside of the basin device comprises a first habitat. The habitat may equal to the complete inside of the basin device or may be only a part of the area enclosed by the basin device.

The lighting device comprises at least one light emitting solid-state lighting source. The solid-state lighting source may comprise a LED-lighting source, an OLED-lighting source, a SSL-Laser lighting source and the like. The lighting device may comprise one single solid-state lighting source or several solid-state lighting sources. The several solid-state lighting sources may all be of the same kind or may be configured individually, e.g. one as an OLED-lighting source, one as a LED-lighting source etc.

The lighting device is adapted for the first species, accommodated in the first habitat. That is the light, emitted by the lighting device has a spectrum optimized for growing said first species. A respective optimization includes a spectrum being optimized with regard to photosynthesis activity of the species, special ambient conditions of the species, light intensity, light wavelength, preventing bacteria infections, preventing fungal infections, heating effect on the environment, penetrability for the environment (e.g. air, water etc.), length of time of illumination etc.

The lighting device illuminates the first habitat and the corresponding species. By this illumination light, energy is transmitted to the species and the habitat. This light energy is adapted to the optimized growing of the species.

The inventive bioreactor apparatus comprises at least a second habitat adapted for a second species. By this way, two different kinds of species may be grown in one single bioreactor apparatus. The second habitat may be illuminated by the first lighting device or may be illuminated by a second lighting device. The second lighting device may be the same as the first lighting device or may be constructed differently. The second lighting device is in one preferred embodiment adapted for the optimum growth of said second species

In a preferred embodiment said bioreactor apparatus comprises at least two basin devices having at least one habitat each. Alternatively, the apparatus comprises at least one basin device provided with at least two habitats. The habitats may be separated from each other by separating walls being inserted into the inside of a respective basin device. The habitats may be isolated from each other or may communicate with each other.

In another embodiment of the present invention said habitats are arranged successively, adopted for successive species of a food chain, wherein the arrangement corresponds to the stage of food chain the species comply to, for creating an artificial food chain.

Therefore, in one embodiment of the present invention a first habitat is adopted for housing species of a primarily stage of a food chain, comprising primary producers and/or autotrophs. Of course the first habitat may be adopted for other species, e.g. for heterotophs. The adoption includes all parameters needed for housing species of a primary stage, e.g. environmental parameters like light, atmosphere and the like. Especially the light environment is adjusted for the primary species. That is the emitted light illuminating the first habitat has a particular spectrum, which is preferably in the range of 200 nm to 1000 nm. More preferably the emitted light is red light, especially red light having a wavelength of about 666 nm. Preferably, the lighting device comprises several LED-lighting sources, each adjusted to emit red light, especially with a wavelength of about 666 nm. The LED-lighting source may be placed directly in the habitat, which for example comprises water as an atmosphere. The primary species serves as food for a species of a successive stage of the food chain.

In another embodiment of the present invention, a second habitat is adopted for housing species of a secondary stage of a food chain, comprising consumers and/or heterotrophs. The adoption includes all parameters needed for housing species of a secondary stage, e.g. environmental parameters like light, atmosphere and the like. Especially, the light environment is adjusted for the secondary species. That is the emitted light illuminating the second habitat has a particular spectrum, which is preferably in the range of 200 nm to 1000 nm. More preferably the emitted light is red combined with some other light, especially red light combined with orange, yellow and/or green light having a wavelength of about 625-520 nm. The lighting device comprises preferably several LED-lighting sources, each adjusted, to emit light from a red spectrum to a green spectrum, especially with a wavelength of about 625-520 nm. The LED-lighting source can be placed directly in the habitat, which for example comprises water as an atmosphere. The secondary species may serve as food for a successive species of the food chain.

In still another embodiment of the present invention a third habitat is adopted for housing species of a third stage of a food chain, comprising heterotrophs, preferably heterotrophs different to said heterotrophs from said second stage. The adoption includes all parameters needed for housing species of a third stage, e.g. environmental parameters like light, atmosphere and the like. Especially, the light environment is adjusted for the third species. That is the emitted light illuminating the third habitat has a particular spectrum, which is preferably in the range of 200 nm to 1000 nm. More preferably the emitted light is green light especially green light having a wavelength of about 520-565 nm. The lighting device comprises preferably several LED-lighting sources, each adjusted, to emit green light, especially with a wavelength of about 540 nm. The LED-lighting source can be placed directly in the habitat, which for example comprises water as an atmosphere. Again, the third species may serve as food for a successive stage of species. Additionally, the lighting device comprises pulsed UV LED-lighting sources, which are installed to treat fungal diseases of the species of a third stage. The third stage may be the last stage in a food chain.

In a further embodiment of the present invention, a last habitat is adopted for housing waste of heterotrophs and the like for recycling said waste. The last habitat, may be the fourth, fifth, etc. habitat of a food chain or the last one of an arrangement of a plurality of successive habitats. The adoption includes all parameters needed for housing species of a last stage, e.g. environmental parameters like light, atmosphere and the like. Especially, the light environment is adjusted for the last species or the corresponding waste. That is the emitted light illuminating the last habitat has a particular spectrum, which is preferably in the range of 200 nm to 1000 nm. The lighting device comprises preferably several LED-lighting sources. The LED-lighting source may be placed directly in the habitat, which for example comprises water as an atmosphere. Additionally, the lighting device may comprise pulsed UV LED-lighting sources, which are installed to treat fungal diseases of the species of a third stage.

In one preferred embodiment of the present invention, the habitats are linked via a connection system having locking means, for connecting and separating the habitats. The habitats may be arranged in one common basin device, separated by separating walls. To connect the separated habitats, the connection system may comprise pipes, conduits and the like, connecting different habitats. The pipes, conduits and the like may be blocked or locked via gates or lockings, so that the habitats may be communicated or separated. The connection system may comprise conveyors, conveying species and/or atmosphere from one habitat to another. Additionally a control unit may be installed, to regulate the conveying.

The habitats may be arranged in separate basin devices, especially spaced basin devices. In this case the connection system may connect the basin devices for a connection of said habitats.

The different habitats are defined at least partly by separating walls. The separating walls may be additional walls in a basin device or may be part of the walls of the basin devices. The separating walls may be plate-like or may have the form of a tube, depending partly on the shape of a respective basin device.

In a further embodiment of the present invention, the solid-state lighting source comprises at least one LED, especially one inorganic LED, one OLED, one laser diode, one optical conductor, connected to the LED, and/or one radiating structure, connected to the optical conductor. The optical conductor and/or the radiating structure are optional and may be added additionally. The use of LEDs has the following advantages:

Firstly, the performance of LEDs/OLEDs is high and still improving rapidly. Secondly, the wavelength of the LEDs/OLEDs light output may be tuned to the absorption spectra of species to achieve best growth rates at low energy consumption. Thirdly, LEDs/OLEDs may be controlled (dimming, pulsing, waveforms, frequency etc.) very easily. Fourthly, LEDs/OLEDs with specific wavelength may be used to eliminate harmful bacteria to treat fungal infections of the species and to purify an atmosphere, e.g. water. Fifthly, LEDs/OLEDs may be easily cooled and generated heat may be reused. Sixthly, OLEDs are very suitable for bioreactor applications because of their excellent form factor.

Using a lighting device for optimized growth makes growing independent of sunlight and/or weather conditions. Especially the light intensity and the spectral conditions can be controlled as needed. Light sources can be brought as near as wanted to the species to realize optimized growth, so that no or nearly no light is absorbed by for example water. By using a small spectrum instead of the full white spectrum the control of a spectral composition of the light can be easily realized for creating optimal conditions for just one type of species or organism. This may avoid competition for nutrients from other species.

By using multiple basin devices, a complete fish food chain and the like may be created and the different species may be controlled separately and optimally by LEDs/OLEDs.

The above mentioned solid-state lighting source is very small and may easily be integrated in basin devices.

In one preferred embodiment of the present invention, the LED, especially said inorganic LED, said OLED, said laser diode, said optical conductor, connected to the LED, and/or said radiating structure, connected to the optical conductor is/are integrated in said separating wall(s) of a basin device or of several basin devices as well as in other walls of the basin device. The corresponding wall is preferably made of a material that conduits light.

In another preferred embodiment of the present invention, the apparatus comprises a radiating structure which is arranged at a surface of said separating wall(s), facing to the corresponding habitat, for illuminating said habitat and its contents. By this arrangement the solid-state lighting source may be integrated in the basin device very easily.

A further embodiment of the present invention comprises a controller for at least controlling said lighting device, such that each solid-state lighting source emits light of a specific spectrum and/or a specific intensity corresponding to the optimum growth of the related species. Solid-state lighting sources may easily be controlled with respect to the emitted light. Therefore, one kind of a solid-stage lighting source may be used as a lighting source for a first habitat as well as a second habitat, wherein the emitted light may have different spectra. The controller may control one or more parameter of the light to be emitted.

In still a further embodiment of the present invention, the bioreactor apparatus further comprises a cooling system for cooling at least partly the lighting device. For an efficient operation the lighting device may be cooled. The heat generated by the lighting device may be reused. For example the produced heat can be used to heat the habitat.

One preferred example comprises a cooling system, which is adopted as an internal cooling system, using the contents of the corresponding habitat for cooling. If the content of a habitat is for example water, the heat generated by the lighting device may be used for heating the water.

In another preferred embodiment of the present invention, the cooling system is adopted as an external cooling system, having conduits for transferring heat outside the contents of the corresponding habitat including the separating walls. In this manner heat could be transferred to a place outside the habitat, where it is needed. Therefore, an exact regulation of the heat in said corresponding habitat may be realized.

In one embodiment of the present invention, the bioreactor apparatus is adopted as an aquatic ecoregion, especially for forming a fish farm, mussel or clam farm, lobster farm and the like. Each habitat may be adopted as aquatic ecoregion, wherein different habitats may build different aquatic ecoregions, e.g. adopted for different species. The aquatic ecoregion may be a marine ecoregion or a freshwater region. In one habitat a marine ecoregion may be established and in another habitat a fresh water ecoregion may be established.

In another embodiment of the present invention, the bioreactor apparatus is adopted as a terrestrial ecoregion, especially for forming a green house. Different habitats may be adopted as different terrestrial ecoregion, for example for different species. Of course, different habitats may be arranged as different ecoregions, e.g. one habitat may be arranged as aquatic ecoregion and another habitat may be arranged as terrestrial ecoregion.

In still another embodiment the bioreactor apparatus comprises emitting devices as UV-LEDs, UV-OLEDs, pulsed UV-LEDs, pulsed UV-OLEDs, and the like, for the treatment of diseases, infections and other optimum growth preventing effects including preventing growth of unwanted species, providing an optimized spectral composition, pulse frequencies, and/or day and night cycle light for the corresponding species. By the use of such emitting devices the optimum growth of one certain species can be realized. No energy is wasted for the growth of a second species in one common basin. The habitat is optimized for growing one certain species, preventing any growth preventing effects, like fungal diseases, bacterial or other infections.

The object of the invention is also achieved by a bioreactor system comprising at least two bioreactor apparatus, one adopted as an aquatic ecoregion, one adopted as a terrestrial ecoregion, both combined to form a complex artificial ecoregion. Each bioreactor apparatus may have different kinds of aquatic and terrestrial ecoregions, respectively.

Further, the object of the invention is also achieved by a method for growing light energy dependant biological species in at least one bioreactor apparatus, the method comprising: illuminating a first species in a first habitat by a first lighting device emitting light having a first wavelength, transferring the grown first species to a successive habitat, separated from the previous habitat, via a connection system, illuminating a successive species in said successive habitat by a successive lighting device emitting light having a different spectrum to the light of the previous lighting device, and repeating the steps transferring and illuminating until the desired species has grown to an optimum.

The aforementioned bioreactor apparatus, as well as claimed components and the components to be used in accordance with the invention in the described embodiments, are not subject to any special exceptions with respect to size, shape, material selection as technical concept such that the selection criteria are known in the pertinent field can be applied without limitations. Additional details, characteristics and advantages of the object of the present invention are disclosed in the dependent claims and the following description of the illustrating figures, showing preferred, but not-limiting embodiments of the present invention, in which:

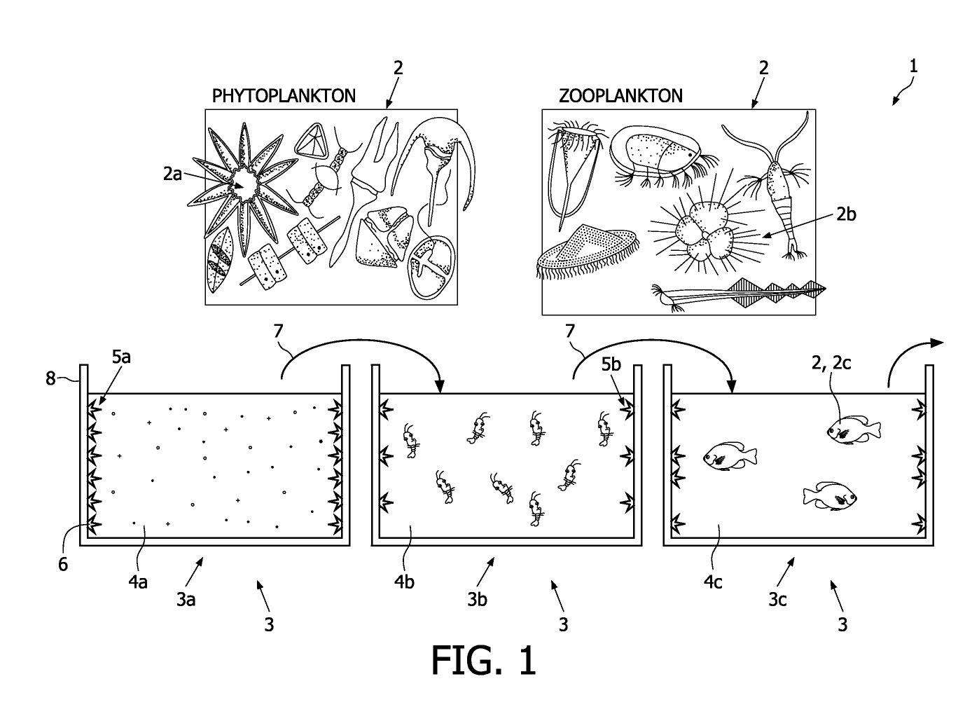

FIG. 1 shows a schematic side view of an embodiment of a bioreactor apparatus,

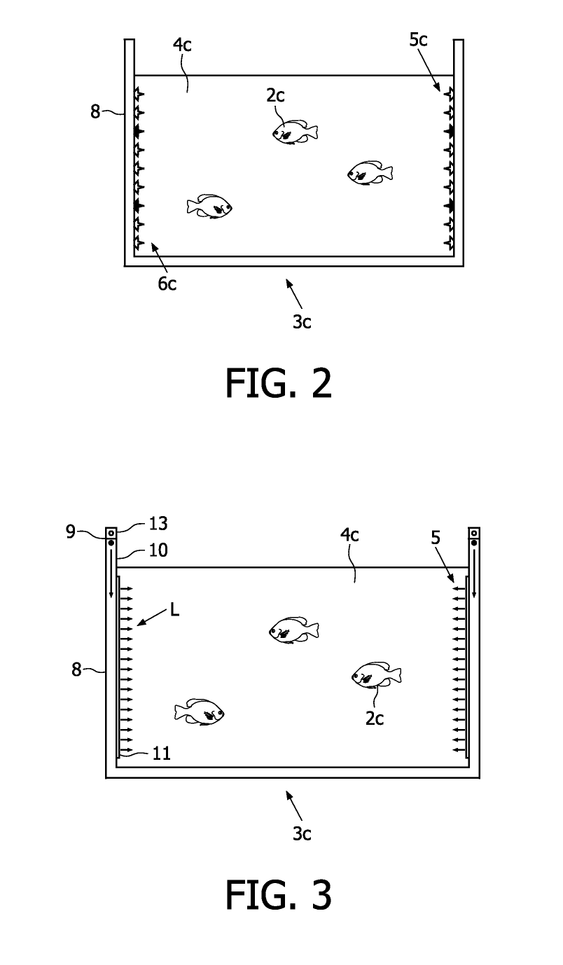

FIG. 2 shows a schematic side view of an embodiment of a basin device,

FIG. 3 shows a schematic side view of another embodiment of a basin device,

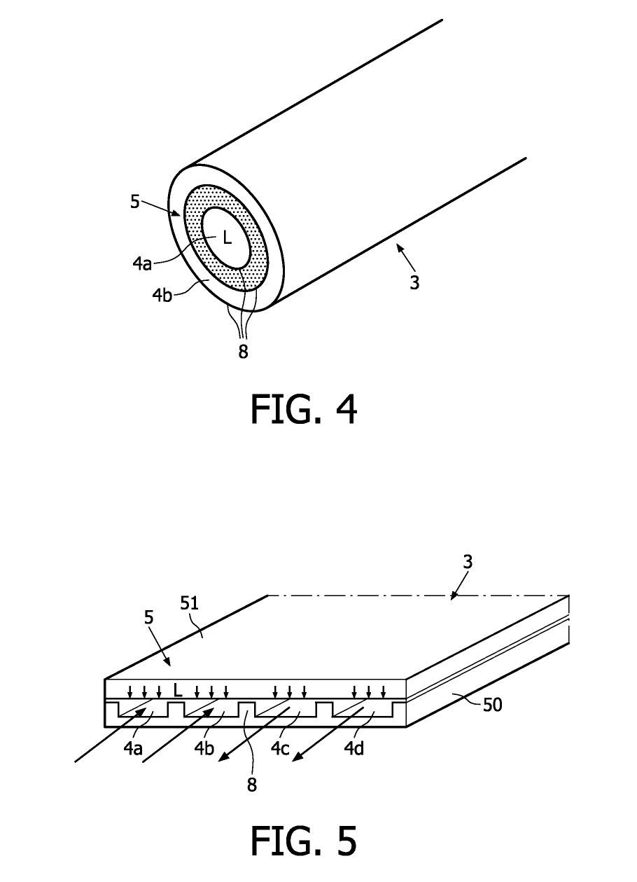

FIG. 4 shows a schematic perspective view of another embodiment of a bioreactor apparatus,

FIG. 5 shows a schematic perspective view of yet another embodiment of a bioreactor apparatus,

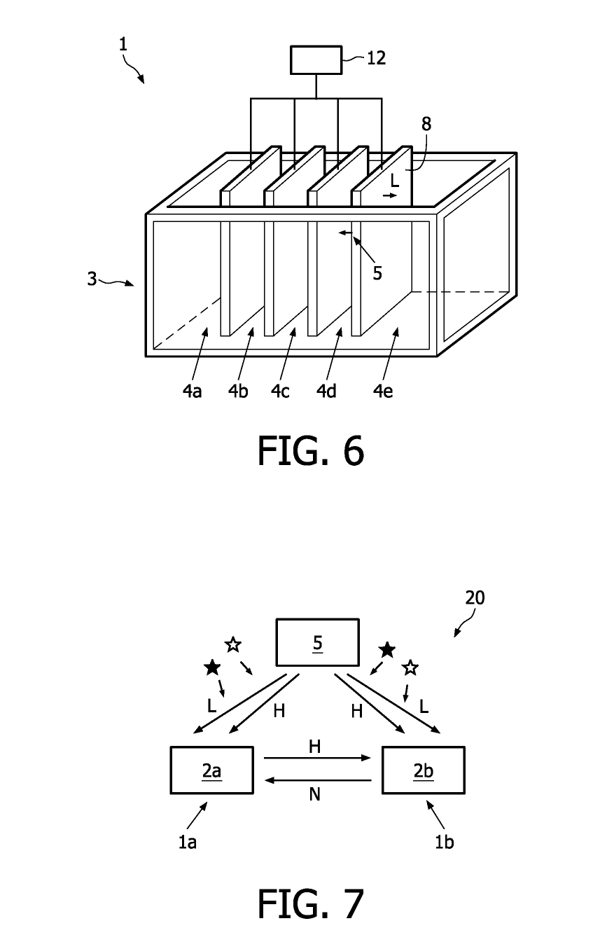

FIG. 6 shows a schematic perspective view of another embodiment of a bioreactor apparatus, and

FIG. 7 shows a schematic bioreactor system with a combination of a green house and a fish farm.

FIG. 1 shows a schematic side view of an embodiment of a bioreactor apparatus 1 for growing different species 2 comprising three basin devices 3. Each basin device 3 is adopted for a different kind of species 2a, 2b, 2c. A first basin device 3a is adopted for accommodating a first species 2a, a second basin device 3b is adopted for accommodating a second species 2b, and a third basin device 3c is adopted for accommodating a third species 2c.

The first species 2a is a primary species of primarily stage of a food chain, comprising primary producers and/or autotrophs like

The second species 2b is a secondary species of a secondary stage of a food chain, comprising consumers and/or heterotrophs.

The third species 2c is a third species of a third stage of a food chain, comprising heterotrophs, especially heterotrophs being different to said heterotrophs of a primarily stage, like the second stage. In this embodiment, the third stage is the last stage of a food chain.

Each basin device 3a-3c defines a habitat 4a-4c in which the respective species 2a-2c is located.

Further, the bioreactor apparatus 1 comprise at least one lighting device 5 in at least one basin device 3. In the example according to FIG. 1, the first basin device 3a comprises a first lighting device 5a, the second basin device 3b comprises a second lighting device 5b, and the third basin device 3c comprises a third lighting device 5c. The lighting devices 5a-5c are arranged differently, wherein each lighting device 5 is adopted according to the species 2 located in the respective basin device 3. The difference in the different lighting devices 5 may be in the kind of lighting source being used or the intensity and/or the spectrum of light the lighting source emits and/or any other parameter.

Each lighting device 5 comprises at least one solid-state lighting source 6. The solid-state lighting source 6 according to FIG. 1 is a LED-lighting source, especially an OLED-lighting source. The depicted lighting sources 6 differ in the different lighting devices 5a-5c. For example, the lighting sources 6 differ in number, in arrangement, in emitted spectrum of light and so on.

The different device basins 3, especially the different habitats 4, are connected via a connection system 7. The connection system is schematically illustrated by an arrow, wherein the arrow represents a tube system or any other conveying system for conveying species and/or other contents of a habitat 4 to another habitat 4. The arrangement of a basin device is shown in the following figures in more detail.

FIG. 2 shows a schematic side view of an embodiment of a basin device 3. The basin device 2 is formed as a block-shaped aquarium, having glass walls or walls made of a light conducting material, defining a habitat 4. The example of FIG. 2 represents a third stage of a food chain according to FIG. 1, therefore, the index “c” is used in most reference numbers. The basin device 3c is adopted for a third species 2c, which in this example are fishes. Therefore, the habitat 4c accommodates water as a suitable surrounding for said fishes. The lighting device 5c comprises several LED-lighting sources 6c, wherein the lighting sources 6c partly differs from each other. Possible differences include an emitted spectrum, a different form, a different intensity and so on. The lighting device 5c is arranged at separating walls 8 of the basin device 3c, which define the habitat 4c of said basin device 3c. In this case, the habitat 4c comprises the complete interior of said basin device 3c and the separating walls 8 coincide to the outer walls of said basin device 3c.

FIG. 3 shows a schematic side view of another embodiment of a basin device 3, wherein the basin device of FIG. 3 represents a third basin device 3c according to FIG. 1. The single solid-state lighting sources 6 are represented by little arrows which also show the main direction of illumination. The basin device 3c is adopted for a third stage species 2c. In particular, the lighting device 5 is adopted for that species 2c, i.e. light (L) is emitted, which is perfectly absorbed by said species 2c for optimum growth. The lighting device 5 comprises at least one LED 9. The LED 9 is arranged adjacent to one separating wall 8 and emits light along an optical conductor 10, which in the depicted example is integrated in one separating wall 8. For emitting the light (L) to the respective habitat 4, the lighting device 5 comprises a radiating structure 11, which is connected to the optical conductor. The radiating structure may be any structure, guiding the light L to the habitat. The generation of said light L can be controlled by a controller 12 (shown in FIG. 6). For using the heat generated by the LED the lighting device 5 comprises a cooling system 13, which may be an internal cooling system or an external cooling system. The cooling system in FIG. 3 is integrated at the separating wall and comprises conduits, transporting the generated heat via a tube system. A suitable heat energy transfer medium may be the water from the habitat 4.

FIG. 4 shows a schematic perspective view of another embodiment of a bioreactor apparatus 1. The bioreactor apparatus 1 comprises a basin device 3. The basin device 3 may have any shape or form. In FIG. 4, the basin device 3 has a kind of tubular form, with two habitats 4a, 4b having an annular cross section in a first direction, separated from each other by a tubular separating wall 8. In other words, a first (cylindrical) habitat 4a is defined by the inside of a tubular separating wall 8, which is surrounded by a spaced cylindrical wall, so that a second (tubular) habitat 4b is formed. In the separating wall 8, being sandwiched by the two habitats 4a, 4b a lighting device 5 is integrated. The lighting device 5 may either illuminate habitat 4a or habitat 4b or both of them simultaneously. The habitats 4a, 4b may be connected by a connection system (not shown in FIG. 4), so that a communication of the contents of both habitats can be realized, wherein for example the contents of habitat 4a flow to the habitat 4b.

FIG. 5 shows a schematic perspective view of yet another embodiment of a bioreactor apparatus 1. The bioreactor apparatus 1 comprises a basin device 3, defining four habitats 4a-4d. The basin device 3 is built of two plate-like parts 50, 51, placed adjacent to each other. At least one of the plate-like parts 50 has grooves and projections, defining cavity for the habitats 4, wherein the habitats 4 are covered by the second part 51. The grooves, which extend in a length-wise direction of the parts 50, 51, may be connected by a connection system 7 (not shown), so that a communication between the different habitats 4 can be realized. The lighting device 5 is integrated in at least on of the two parts, defining the basin device 3.

FIG. 6 shows a schematic perspective view of another embodiment of a bioreactor apparatus 1. The bioreactor apparatus 1 comprises one basin device 3. The basin device 3 is formed as a block-shaped aquarium, having four separating walls 8 in its interior. The habitats 4 are separated from each other by the separating walls 8 and the outer walls of the basin device 3. In this example, each separating wall 8 has an integrated lighting device 5. The lighting device 5 can emit light L in at least two directions that is in two different habitats 4. For an adopted emission of light L according to the respective species, the lighting devices 5 are controlled by a controller 12. The controller 12 controls spectrum, wavelength, intensity, lighting-time, lighting-frequency and the like.

FIG. 7 shows a schematic bioreactor system 20 of a combination green house and fish farm. The green house represents a first bioreactor apparatus 1a and the fish farm represents a second bioreactor apparatus 1b. The interdependencies of the two bioreactor systems 1a and 1b with regard to the lighting device 5, heat H, nutrition N, and light L is shown.

The first bioreactor 1a, which comprises a first species 2a receives light L and heat H emitted by the lighting device 5. The first species 2a can be plants and the like. The first bioreactor apparatus 1a transfers heat H to the second bioreactor apparatus 2b. The second bioreactor apparatus 2b also receives heat H and light L from the lighting device 5.

The lighting device 5 is adopted, to optimize the growth of the two species 2a, 2b. First species 2a serves as food for species 2b. Species 2b may be a supply for species 2a. So a complex ecoregion can be realized. Preferably, the first bioreactor apparatus 1a is a terrestrial ecoregion and the second bioreactor apparatus 1b is an aquatic ecoregion.

The bioreactor apparatus 1 may comprise the following components:

at least one water basin to grow at least one species of a fish food chain; at least one species of a fish food chain; at least one organic (OLED) and/or inorganic light emitting diode (LED).

Additionally the following features may be added: at least one cooling system for the OLEDs/LEDs, at least one basis, which uses sunlight; other light sources, such as HPS lamps, wherein LEDs or OLEDs may be used to produce light with missing wavelength(s); a controller to guarantee a constant light output independent of the changes of the sunlight, and/or a control system for OLEDs/LEDs, wherein the control system may control: light (intensity, on/off, pulsing, day-night cycle, spectrum, . . . ); water temperature; concentration of water additive (nutrition, medical fluids, minerals, . . . ); CO₂ concentration in the water; movement/circulation of the water.

The bioreactor system 20 comprises for example the following components:

a green house, breeding beds for plants including plants in the greenhouse, an algae and/or fish basin device 3 for growing algae and/or fish in the greenhouse, a first lighting device 5, preferably comprising HPS lamps, a second lighting device 5 comprising LEDs/OLEDs located in the greenhouse for optimal plant growth and/or in the water of the basin device 3 for optimal fish/algae growth.

The fish basin device 3 can be a fish farm or can be a different bioreactor.

The fish/algae reuse the light in the greenhouse as well as the heat generated by the lighting device 5. Alternatively, the fish/algae may also be used for other purposes such as bio oil, pigments and proteins for medicines, and proteins for the cosmetic industry. The combined system offers the following advantages:

he fish enrich the water for the algae/plants with nutrition (N, P, C, . . . ). The water is used and not thrown away. Energy is ideally used. The origin of both, fish and plant, is easy to track, which increases the safety. Space is used optimally. Water is double used.

### LIST OF NUMERALS

1 bioreactor apparatus
2 species
2a first species
2b second species
2c third species
3 basin device
3a first basin device
3b second basin device
3c third basin device
4 habitat
4a first habitat
4b second habitat
4c third habitat
4d fourth habitat
4e fifth habitat
5 lighting device
5a first lighting device
5b second lighting device
5c third lighting device
6 solid-state lighting source(s)
7 connection system
8 separating wall(s)
9 LED
10 optical conductor
11 radiating structure
12 controller
13 cooling system
20 bioreactor system
50 (first plate-like) part
51 (second plate-like) part
L Light
H heat
N nutrition

1. A bioreactor apparatus (1) for growing of light energy dependant biological species (2),
comprising at least one basin device (3) defining a first habitat (4a) for accommodating a first species (2a), and
a first lighting device (5a) having at least one light emitting solid-state lighting source (6), adapted for the first species (2a) by emitting light (L),
wherein the solid-state lighting source (6) illuminates said habitat (4) using light energy emitted from the solid-state lighting source (6) to said first species (2a) characterized in that,
the bioreactor apparatus (1) comprises at least a second habitat (4b), adapted for a second species (2b), for growing at least two different kinds of species (2a,2b) in one bioreactor system.

2. A bioreactor apparatus (1) according to claim 1,
characterized in that the apparatus (1) comprises at least two basin devices (3a,3b,3c) having at least one habitat (4) each.

3. A bioreactor apparatus (1) according to claim 1 or 2,
characterized in that the apparatus (1) comprises at least one basin device (3) provided with at least two habitats (4).

4. A bioreactor apparatus (1) according to any preceding claims,
characterized in that the habitats (4) are arranged successively, adopted for successive species (2) of a food chain, wherein the arrangement corresponds to the stage of food chain the species (2) comply to, for creating an artificial food chain.

5. A bioreactor apparatus (1) according to any preceding claims,
characterized in that a first habitat (4a) is adopted for housing species (2a) of a primarily stage of a food chain, comprising primary producers and/or autotrophs.

6. A bioreactor apparatus (1) according to any preceding claims,
characterized in that a second habitat (4b) is adopted for housing species (2b) of a secondary stage of a food chain, comprising consumers and/or heterotrophs.

7. A bioreactor apparatus (1) according to any preceding claims,
characterized in that a third habitat (4c) is adopted for housing species (2c) of a third stage of a food chain, comprising heterotrophs.

8. A bioreactor apparatus (1) according to any preceding claims,
characterized in that a last habitat (4d) is adopted for housing waste of heterotrophs the like for recycling said waste.

9. A bioreactor apparatus (1) according to any preceding claims,
characterized in that the habitats (4) are linked via a connection system (7) having locking means, for connecting and separating the habitats (4).

10. A bioreactor apparatus (1) according to any preceding claims,
characterized in that the habitats (4) are defined at least partly by separating walls (8).

11. A bioreactor apparatus (1) according to any preceding claims,
characterized in that the solid-state lighting source (6) comprises at least one LED (9), especially one inorganic LED, one OLED, one laser diode, one optical conductor (10), connected to the LED, and/or one radiating structure (11), connected to the optical conductor (10).

12. A bioreactor apparatus (1) according to any preceding claims,
characterized in that the LED (9), especially said inorganic LED, said OLED, said laser diode, said optical conductor (10), connected to the LED, and/or said radiating structure (11), connected to the optical conductor (10) is/are integrated in said separating wall(s) (8).

13. A bioreactor apparatus (1) according to any preceding claims,
characterized in that the said radiating structure (11) is arranged at a surface of said separating wall(s) (8), facing to the corresponding habitat (4), for illuminating said habitat (4) and its contents.

14. A bioreactor apparatus (1) according to any preceding claims,
characterized in that the bioreactor apparatus (1) further comprises a controller (12) for at least controlling said lighting device (5), such that each solid-state lighting source (6) emits light (L) of a specific spectrum and/or a specific intensity corresponding to the optimum growth of the related species (2).

15. A bioreactor apparatus (1) according to any preceding claims,
characterized in that the bioreactor apparatus (1) further comprises a cooling system (13) for cooling at least partly the lighting device (5).

16. A bioreactor apparatus (1) according to any preceding claims,
characterized in that the cooling system (13) is adopted as an internal cooling system, using the contents of the corresponding habitat (4) for cooling.

17. A bioreactor apparatus (1) according to any preceding claims,
characterized in that the cooling system (13) is adopted as an external cooling system, having conduits for transferring heat outside the contents of the corresponding habitat (4) including the separating walls (8).

18. A bioreactor apparatus (1) according to any preceding claims,
characterized in that the bioreactor apparatus (1) is adopted as an aquatic ecoregion, especially for forming a fish farm, mussel or clam farm, lobster farm and the like.

19. A bioreactor apparatus (1) according to any preceding claims,
characterized in that the bioreactor apparatus (1) is adopted as a terrestrial ecoregion, especially for forming a green house.

20. A bioreactor apparatus (1) according to any preceding claims,
characterized in that the bioreactor apparatus (1) comprises emitting devices as UV-LEDs, UV-OLEDs, pulsed UV-LEDs, pulsed UV-OLEDs, and the like, for the treatment of diseases, infections and other optimum growth preventing effects including preventing growth of unwanted species, providing an optimized spectral composition, pulse frequencies, and/or day and night cycle light for the corresponding species.

21. A bioreactor system (20) comprising at least two bioreactor apparatuses (1a,1b), one adopted as an aquatic ecoregion, one adopted as a terrestrial ecoregion, both combined to form a complex artificial ecoregion.

22. A method for growing light energy dependant biological species (2) in at least one bioreactor apparatus (1), the method comprising:
illuminating a first species (1) in a first habitat (4a) by a first lighting device (5a) emitting light (L) having a first spectrum,
transferring the grown first species (2a) to a successive habitat (4b, 4c, 4d, . . . ), separated from the previous habitat (4), via a connection system (7),
illuminating a successive species (2b,2c, . . . ) in said successive habitat (4b,4c,4d, . . . ) by a successive lighting device (5b,5c, . . . ) emitting light (L) having a different spectrum to the light (L) of the previous lighting device (5), and
repeating the steps transferring and illuminating until the desired species (2) has grown to an optimum.
